(19) United States
(12) Patent Application Publication
(10) Pub. No.: US 2024/0090949 A1
(43) Pub. Date: Mar. 21, 2024

(54) SYSTEM AND METHOD FOR MINIMALLY INVASIVE SURGICAL INTERVENTIONS

(71) Applicant: SYNAPTIVE MEDICAL INC., TORONTO (CA)

(72) Inventors: Cameron PIRON, Toronto (CA); Ian SWANSON, Toronto (CA); Thanh VUONG, Kitchener (CA)

(73) Assignee: SYNAPTIVE MEDICAL INC., Toronto (CA)

(21) Appl. No.: 18/471,682

(22) Filed: Sep. 21, 2023

Related U.S. Application Data
(60) Provisional application No. 63/408,517, filed on Sep. 21, 2022.

(51) Int. Cl. A61B 34/10; A61B 34/00; A61B 34/20; A61B 90/00

(52) U.S. Cl. CPC A61B 34/10; A61B 34/20; A61B 34/25; A61B 90/37; A61B 2034/2051; A61B 2034/254; A61B 2090/374

(57) ABSTRACT

A system and methods for supporting minimally invasive surgery, involving a control module configurable to: receive an initial MRI image from an MRI imaging device; transmit the initial MRI image to a planning module configured to determine a surgical plan; receive the surgical plan from the planning module; transmit the surgical plan to a guidance module configured to operate with a medical instrument and the MRI imaging device, the medical instrument configured to couple with an MRI micro-coil, and the MRI imaging device configured to operate with the MRI micro-coil; and receive, in real-time, a subsequent MRI image from the MRI imaging device operating with the MRI micro-coil during guidance of the medical instrument.

### CROSS-REFERENCE TO RELATED APPLICATION(S)

This document is a nonprovisional patent application claiming the benefit of, and priority to, U.S. Provisional Patent Application Ser. No. 63/408,517, filed on Sep. 21, 2022, entitled “System and Method for Minimally Invasive Surgical Interventions” and hereby, incorporated by reference in its/their entirety.

### FIELD

The present disclosure relates to systems and methods for magnetic resonance imaging (“MRI”) and minimally invasive surgery.

### BACKGROUND

Minimally invasive procedures produce less trauma to patients as opposed to open surgery, thereby resulting in improved outcomes while enabling more patients to receive care. Minimally invasive procedures are limited today by a lack of imaging and technology that allows such procedures to be safely performed.

In the related art, deep brain stimulation (DBS) represents a growing procedure in terms of volume and indication for today's neurosurgical programs. With the growing demand, hospitals purchasing new stereotactic guidance systems are increasingly seeking platforms with DBS capabilities in addition to standard cranial and spine indications. The lack of DBS capabilities limits the market opportunity of any stereotactic navigation system today.

Currently, DBS surgical procedures are complex and highly technical with lengthy operating room (OR) times and limited profitability for hospitals. The DBS procedures are further challenged by accuracy of electrode placement (one of ten electrodes are inaccurately placed and require a recurrent surgery) and a need to perform these DBS surgeries with the patient being awake (95% of DBS surgery being performed while the patient is awake), such challenges further complicating these DBS procedures.

Also, the gold-standard” for targeting tissue and diagnosis of pathology is MRI in the related art; however, guidance is typically performed by using navigation techniques based on electrical stimulation information that has been recorded from previously acquired scans. For example, Medtronic® relies on additional imaging, e.g., via an “O-arm” and computerized tomography (CT), to validate location of a medical instrument, such additional imaging having limited soft-tissue contrast. This related art approach is plagued with inefficiency, inaccuracy, and inability to simultaneously validate locations of both the soft tissue target and DBS probe. The technologies in the related have been unable to achieve real-time guidance while also using the gold-standard modality, for at least the following challenges: siting challenges, accuracy concerns, and device incompatibility, e.g., susceptibility and heating.

Further, X-ray imaging with robotics is a focus in the current market, e.g., a MAZOR® robotics system involving integration with surgical navigation and imaging from an O-ARM™ provides a solution to some related art problems, but the MAZOR® robotics system is based on x-ray imaging which is incapable of imaging soft tissue. Therefore, a need exists in the related art, for a system and methods that better solutions to the foregoing related art challenges in relation to surgeries involving soft tissue.

### SUMMARY

The present disclosure addresses at least many of the foregoing challenges experienced by related art in relation to surgery involving soft tissue. The subject matter of the present disclosure generally relates to systems and methods for improving planning, guidance, navigation, and imaging for minimally invasive surgery. The system and methods of the present disclosure further involve using improved surgical planning software and workflow for specific DBS procedures, the surgical planning software and workflow utilizing tractography algorithms that are improved over the related art. The system of the present disclosure is configured for use with an MRI device by utilizing coils, e.g., MRI micro-coils, disposed in relation to medical tools, e.g., surgical tools, for intraoperative guidance during a DBS procedure, whereby at least one of planning, guidance, navigation, and imaging are improved, and whereby real-time, high-definition, MRI images are producible. The systems and methods of the present disclosure involve seamlessly integrating a plurality of technologies, such as planning, diffusion tensor imaging (DTI), MRI, and robotics, into a solution to at least the problems experienced in the related art.

In accordance with an embodiment of the present disclosure, an integrated system for supporting minimally invasive surgery comprises a control module, configurable by a set of executable instructions storable in relation to a non-transient memory device, to: receive at least one initial MRI image of anatomy from an MRI imaging device; transmit the at least one initial MRI image to a planning module configured to determine a surgical plan by using a tractography algorithm; receive the surgical plan from the planning module; transmit the surgical plan to a guidance module configured to operate with a medical instrument and the MRI imaging device, the medical instrument configured to couple with at least one MRI micro-coil, and the MRI imaging device configured to operate with the at least one MRI micro-coil; and receive, in real-time, at least one subsequent MRI image of the medical instrument in relation to the anatomy from the MRI imaging device operating with the at least one MRI micro-coil during guidance of the medical instrument.

In accordance with another embodiment of the present disclosure, a method of providing an integrated system for supporting minimally invasive surgery comprises providing a control module, configurable by a set of executable instructions storable in relation to a non-transient memory device, to: receive at least one initial MRI image of anatomy from an MRI imaging device; transmit the at least one initial MRI image to a planning module configured to determine a surgical plan by using a tractography algorithm; receive the surgical plan from the planning module; transmit the surgical plan to a guidance module configured to operate with a medical instrument and the MRI imaging device, the medical instrument configured to couple with at least one MRI micro-coil, and the MRI imaging device configured to operate with the at least one MRI micro-coil; and receive, in real-time, at least one subsequent MRI image of the medical instrument in relation to the anatomy from the MRI imaging device operating with the at least one MRI micro-coil during guidance of the medical instrument.

In accordance with yet another embodiment of the present disclosure, a method of supporting minimally invasive surgery by way of an integrated system, the method comprising: providing a control module, configurable by a set of executable instructions storable in relation to a non-transient memory device, to: receive at least one initial MRI image of anatomy from an MRI imaging device; transmit the at least one initial MRI image to a planning module configured to determine a surgical plan by using a tractography algorithm; receive the surgical plan from the planning module; transmit the surgical plan to a guidance module configured to operate with a medical instrument and the MRI imaging device, the medical instrument configured to couple with at least one MRI micro-coil, and the MRI imaging device configured to operate with the at least one MRI micro-coil; and receive, in real-time, at least one subsequent MRI image of the medical instrument in relation to the anatomy from the MRI imaging device operating with the at least one MRI micro-coil during guidance of the medical instrument; and activating the integrated system.

Some of the features in the present disclosure are broadly outlined in order that the section entitled Detailed Description is better understood and that the present contribution to the art may be better appreciated. Additional features of the present disclosure are described hereinafter. In this respect, understood is that the present disclosure is not limited in its application to the details of the components or steps set forth herein or as illustrated in the several figures of the being carried out in various ways. Also, understood is that the phraseology and terminology employed herein are for the purpose of the description and should not be regarded as limiting.

### BRIEF DESCRIPTION OF THE DRAWING

The above, and other, aspects, features, and advantages of several embodiments of the present disclosure will be more apparent from the following Detailed Description as presented in conjunction with the following several figures of the Drawing.

FIG. 1 is a display screenshot illustrating tractography, in relation to a patient's brain having a target, used in an epilepsy case, to show “hot” stereoelectroencephalography (sEEG) contacts and their position relative to the tractography for surgical planning, operable by a planning module of an integrated system;

FIG. 2 is a display screenshot illustrating an “Align to Trajectory” feature, operable by a guidance module of an integrated system;

FIG. 3 is a schematic diagram illustrating work-flow of an integrated suite intervention loop, performable by a control module of an integrated system;

FIG. 4 is a diagram illustrating a perspective view of an MRI device configurable for use with an integrated system;

FIG. 5 is a display screenshot illustrating pulse sequence scans from an MRI device configured for use with an integrated system;

FIG. 6 is a graph illustrating protocols for sound pressure levels (SPLs) or acoustic levels in units of A-weighted decibels (dbA), as a function of imager field strength in units of Tesla (T), within a bore of an MRI device;

FIG. 7 is a display screenshot illustrating a plurality of scans comprising a three-dimensional/susceptibility-weighted scan and a tractography scan;

FIG. 8A is a diagram illustrating an MRI image of surgical instrument acquired by using only a head-coil and a field strength of 1.5 T;

FIG. 8B is a diagram illustrating an MRI image of surgical instrument acquired by using only a local, 2-channel, coil and a field strength of 1.5 T;

FIG. 9 is a schematic diagram illustrating an integrated system for supporting minimally invasive surgery;

FIG. 10 is a flow diagram illustrating a method of providing an integrated system for supporting minimally invasive surgery;

FIG. 11 is a flow diagram illustrating a method of supporting minimally invasive surgery by way of an integrated system;

FIG. 12 is a diagram illustrating a perspective view of an MRI device operable with an integrated system;

FIG. 13 is a diagram illustrating a top view of an MRI device operable with an integrated system; and

FIG. 14 is a diagram illustrating a cutaway perspective view of an MRI device operable with an integrated system.

Corresponding reference numerals or characters indicate corresponding components throughout the several figures of the Drawing. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood, elements that are useful or necessary in commercially feasible embodiments are often not depicted in order to facilitate a less obstructed view of these various embodiments of the present disclosure.

### DETAILED DESCRIPTION

In general, the system and methods of the present disclosure involve using hardware and software for supporting minimally invasive surgical interventions. The integrated system uses an MRI device to produce high-definition MRI images by utilizing “DBS” coils disposed in relation to surgical tools during an minimally invasive surgical procedure.

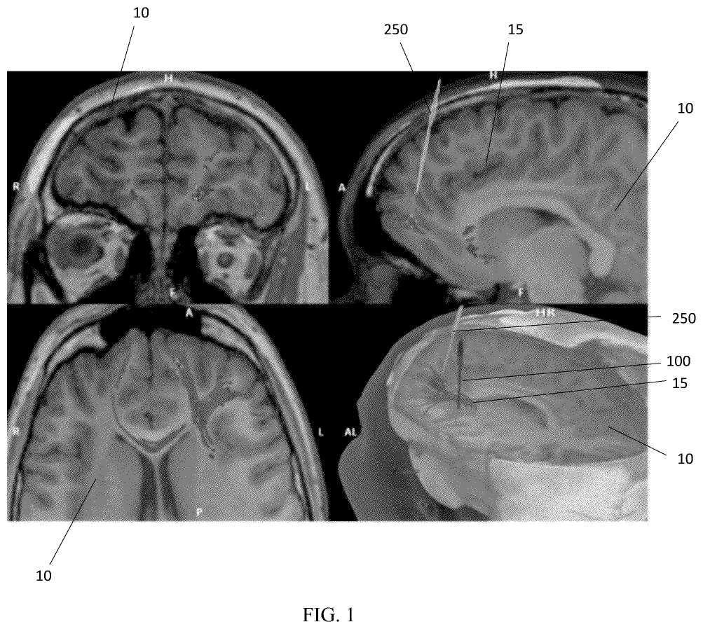

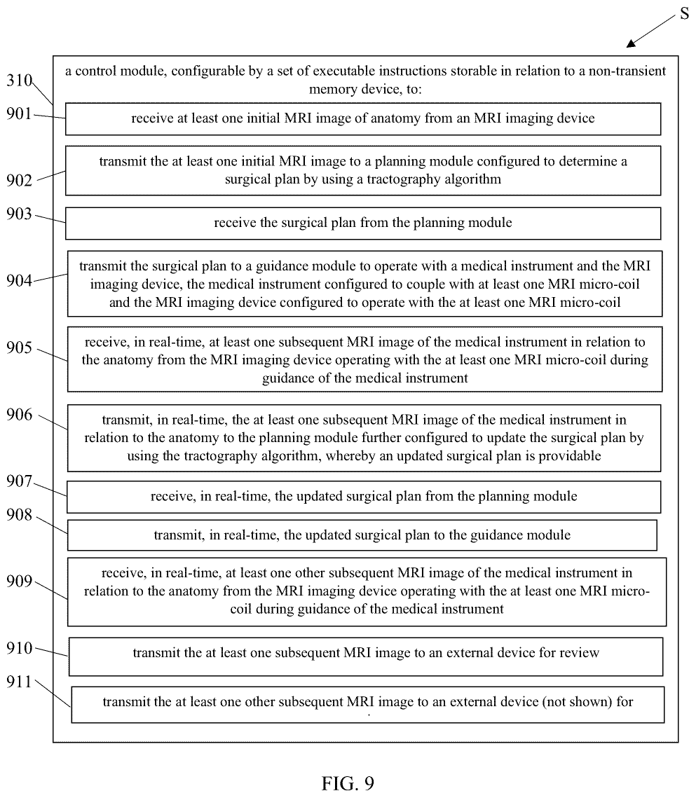

Referring to FIG. 1, this display screenshot illustrates tractography 100 in relation to a patient's brain 10 having a target 15, used in an epilepsy case, for example, to show “hot” sEEG contacts and their position relative to the tractography for surgical planning, operable by a planning module of an integrated system S (FIG. 9), in accordance with an embodiment of the present disclosure. This is an example of applying tractography in functional cases.

### Modus Plan™

Sill referring to FIG. 1, a Modus® Plan™ comprises a software package, operable by a planning module, involving using tractography in medical procedures, such as neurosurgical procedures. Many hospitals consider the Modus Plan™ software package, e.g., operable via a planning module, as the standard of care; and use this software package for all cranial procedures. The Modus Plan™ software package provides automated processing and, thus, provides easy access and data consistency. Further, clinical surgeons have used tractography from the Modus Plan™ software package in their decision making. These advantages extend beyond resection planning to minimally invasive surgery (MIS), e.g., minimally invasive surgical interventions, including DBS.

Sill referring to FIG. 1, the use of cryptography, via the Modus Plan™ software package, in DBS has increased in the last few years; and use of tractography has led to improved surgical outcomes, thereby improving visualization of target areas and, therefore, improving placement of DBS electrodes, e.g., of a DBS probe, thereby reducing the need for repositioning thereof, thereby reducing OR time, and thereby improving clinical outcome. DBS acts at the site of application as well as at a distal location. In embodiments of the present disclosure, the use of DTI to identify targets is useful as DTI facilitates visualization of the tracts (neural tracts) through which stimulation, e.g., via DBS, is transmittable. DTI may enable surgeons to improve targeting accuracy, predict a pattern of activation, and provide insight into potential damage to certain regions with increased stimulation.

Sill referring to FIG. 1, the subject matter of the present disclosure also relates to integration of a plurality of technologies, comprising tractography, for use in surgical planning and for use in an OR environment. The plurality of technologies further comprises DBS operable via smart algorithms based on clinical experience, and optionally, intraoperatively updated, in real-time. For example, the smart algorithms, operable by a planning module, comprise: generating a targeting algorithm, based on a specific medical procedure, such as a DBS procedure and/or any other medical procedure, e.g., involving soft tissue and requiring “high-precision” surgery; optionally, intraoperatively updating, in real-time, the tractography algorithm based on the targeting algorithm; and optionally, intraoperatively updating, in real-time, a tractography algorithm based on research data from, or obtained with, at least one clinical partner.

### Modus V™ Robotics

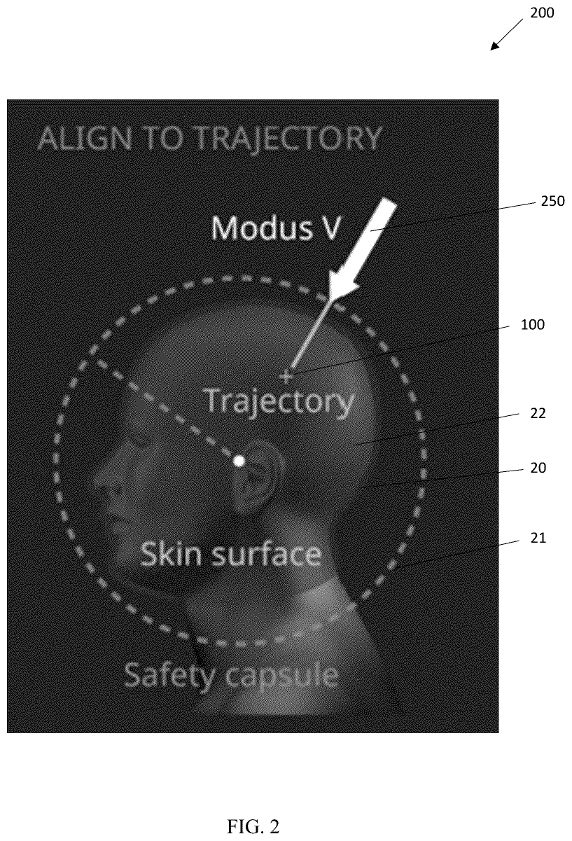

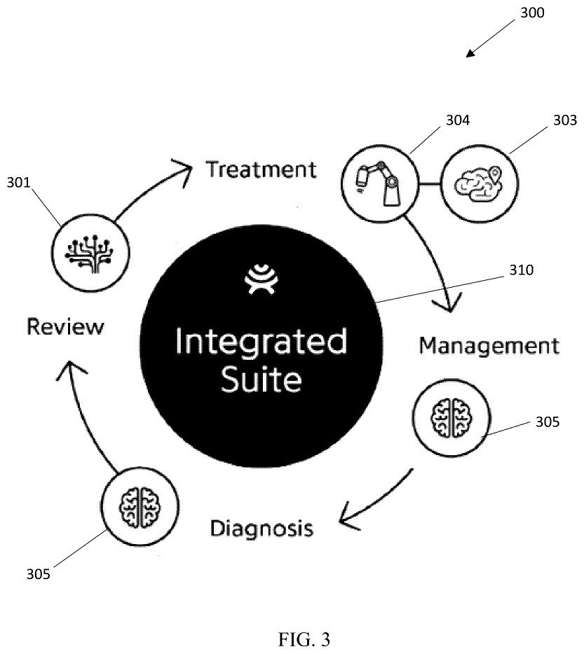

Referring to FIG. 2, this display screenshot illustrates an “Align to Trajectory” feature 200, operable by a guidance module of an integrated system S (FIG. 9) and shown in relation to a side view representation of a patient head 20, in accordance with an embodiment of the present disclosure. Using the integrated system S comprises adapting a robotic digital microscope (not shown) having a camera end effector (not shown), such as a Modus V™ robotic digital microscope system 304 (FIG. 3), to provide a frameless robotic solution for image-guided stereotaxic placement of DBS electrodes, e.g., of a DBS probe 250. Adapting the robotic digital microscope comprises at least one of replacing a robotic arm with a smart robotic arm and replacing the camera end effector with a robotic lead extending end effector (not shown), e.g., a smart end effector (not shown), whereby stereotactic accuracy of tool placement is refined and improved over related art robotic platforms for DBS, e.g., Mazor Renaissance® robotic platform and the Zimmer Rosa® robotic platform. In addition, the robotic lead extending end effector is configured to facilitate additional robotically directed functional operations, such as laser interstitial thermal therapy (LITT) and other ablation procedures.

Still referring to FIG. 2, the integrated system S (FIG. 9), using the smart end effector, provides integration with image-guided tools (not shown), whereby more accurate and seamless DBS workflow is provided over the related art. Replacing the robotic arm with the smart robotic arm comprises using robotic technology, wherein the smart robotic arm of the adapted Modus V™ robotic digital microscope system effects accurate alignment of a payload, such as an optic payload, e.g., an imaging device, that is used in precision neurosurgery (for example, as shown in the right-side images of FIG. 1). The smart robotic arm is operable by the integrated system S, via a guidance system, for guiding an instrument, for example, for effecting accurate placement of DBS electrodes.

Still referring to FIG. 2, the integrated system S (FIG. 9) is configured to instruct operation of the smart robotic arm, e.g., of the adapted Modus V™ robotic digital microscope system, via the guidance module, to align an instrument with a pre-planned surgical trajectory, e.g., a trajectory 100, via the planning module, in coordination with a navigation module, e.g., a tractography-based navigation module which uses geometry data defining at least one of a “safety capsule” 21 and a skin surface 22 of a patient. The integrated system S provides surgeons with a seamless communication among the plurality of technologies, each technology providing distinct information, thereby effecting more accurate and efficient electrode placement over the related art.

### MRI Integrated Suite

Referring to FIG. 3, this a diagram illustrating work-flow of an integrated suite intervention loop 300, such as an MRI integrated suite, performable by a control module 310 of an integrated system S (FIG. 9), in accordance with an embodiment of the present disclosure. The integrated system S provides solutions to significant challenges in the field of MRI intervention, especially in relation to DBS probe delivery. The integrated system S provides benefits to medical procedures, such as DBS procedures, at multiple phases of intervention, such as diagnosis, review, treatment, and management. The control module 310, of the integrated system S, operates at least one of the planning module 301, the navigation module 302, and the guidance module 303, by way of the integrated suite intervention loop 300. To close the integrated suite intervention loop 300, a 0.5 T mid-field MRI device (not shown), for example, is used for pre-imaging and post-imaging a DBS probe, whereby a step-wise approach provides real-time device guidance and has higher accuracy than related art techniques.

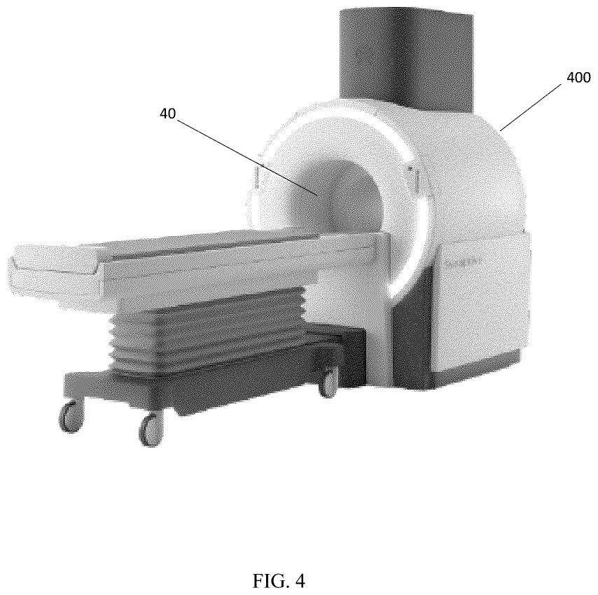

Referring to FIG. 4, this diagram illustrates, in a perspective view, an MRI device 400 configurable for use with an integrated system S, in accordance with an embodiment of the present disclosure. An MRI system is one example of the MRI device 400. The MRI device 400 has a bore 40.

### Phase 1: MRI Device Scans for Targeting and Validation

Still referring to FIG. 4, the MRI device 400, e.g., the MRI device, operable by way of the integrated system S, scans images that are registered with additional preoperative images, as well as to intraoperative images, such as from CT scans, e.g., from Mobius® CT scans, to validate lead placements, in accordance with an embodiment of the present disclosure. A smaller foot-print MRI device could also enable access for imaging in-line with the procedure work-flow, as the MRI device provides an image quality suitable for high-accuracy stereotactic procedures.

Referring to FIG. 5, this display screenshot illustrates pulse sequence scans from an MRI device 400, configured for use with an integrated system S, in accordance with an embodiment of the present disclosure. The MRI device 400, e.g., the MRI device, is configured to scan images by using at least one pulse sequence and using DTI with reduced distortion, whereby targeting accuracy is improved over the related art. The at least one pulse sequence comprises at least one of: a two-dimensional (2D) sequence, a three-dimensional (3D) sequence, at least one advance sequence, such a spin lattice relaxation time (T1) sequence, a transverse relaxation (T2) sequence, a transverse relaxation fluid attenuated inversion recovery (T2 FLAIR) sequence, a diffusion-weighted imaging (DWI) sequence, a magnetic resonance angiography (MRA) sequence, a perfusion sequence, and an susceptibility weighted imaging (SWI) sequence.

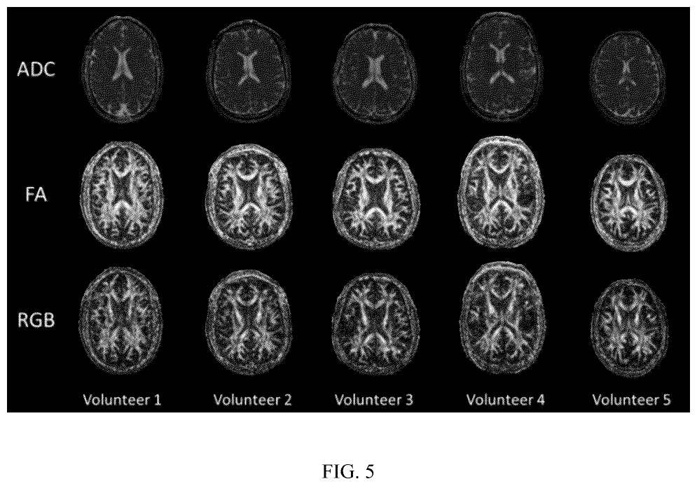

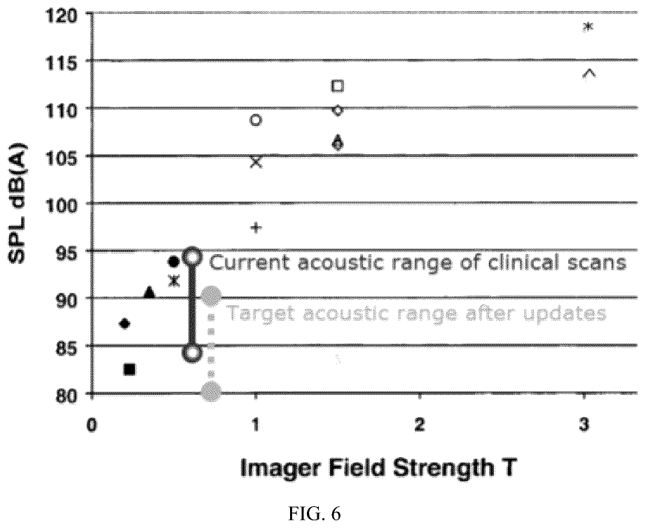

Referring to FIG. 6, this graph illustrates protocols for sound pressure levels (SPLs) or acoustic levels in units of A-weighted decibels (dbA), as a function of imager field strength in units of Tesla (T), within a bore 40 of an MRI device 400, in accordance with an embodiment of the present disclosure. The current protocols for the MRI device 400, e.g., the MRI device, comprises acoustic levels in a range of approximately 84 dbA to approximately 94 dbA within the bore 40. For achieving improved scanning, such as intraoperative imaging, quieter scanning is critical for DBS where 95% of the procedures are performed while the patient is awake.

### Phase 2: Adapted MRI Device—Direct Probe Delivery Under Real-Time MRI Guidance

Still referring to FIG. 6, the integrated system S (FIG. 9) operates to provide direct, real-time, delivery of the probe, e.g., a DBS probe 250 (FIG. 2), to MRI-visible targets. The integrated system S is configured to universally operate with a plurality of distinct DBS probes, such as those manufactured by various major DBS probe vendors. The integrated system S is configured to validate each distinct probe of the plurality distinct probes, whereby low-distortion imaging of each distinct electrodes of each distinct DBS probe is readily visible and distinguishable from imaging of deep brain targets, comprising soft-tissue. The integrated system S is further configured to direct guidance of the probe, e.g., probe 250, in real-time, while directing imaging by the MRI device 400, wherein real-time direct visualization is provided.

Still referring to FIG. 6, the MRI device 400 is configured to adjust its components in relation to the top of the magnet thereof in order to provide additional space for providing better access to the patent head than is otherwise available in the related art. The integrated system S is configured to universally operate a plurality of distinct guidance systems, via the guidance module 303. The plurality of distinct guidance systems comprises guidance systems, such as a ClearPoint® guidance system and, preferably, a Modus V™ guidance system. The integrated system S is configured to universally operate each distinct guidance system of a plurality of distinct guidance systems, via the guidance module 303, to guide a medical instrument, e.g., a probe 250, in real-time, while watching and/or imaging the positioning of the medical instrument, e.g., the probe 250, whereby direct, real-time, visualization is provided, and whereby imaging artifacts of a probe tip are reduced, e.g., approximately nine times less than by using related art 1.5-T MRI systems and approximately thirty-six times less than related art 3.0-T MRI systems, and whereby highly specific probe placement is facilitated.

Still referring to FIG. 6, the MRI device 400 is configured to adjust its components and their operation based on changes in work-flow and ergonomics. The MRI device 400, e.g., the MRI device, is configured to operate with a plurality of scanning protocols, such as a 3D protocol, a gradient echo (GRE) protocol, a T2 protocol, an SWI protocol, and a DWI protocol. The MRI device 400, e.g., the MRI device, is configured to operate with and imaging architecture, in real-time, the imaging architecture synchronized with the placement of the MRI device 400 and a robotic imaging system, such as the Modus V™ robotic digital microscope system 304 having a reconfigured end-effector, such as a smart end effector.

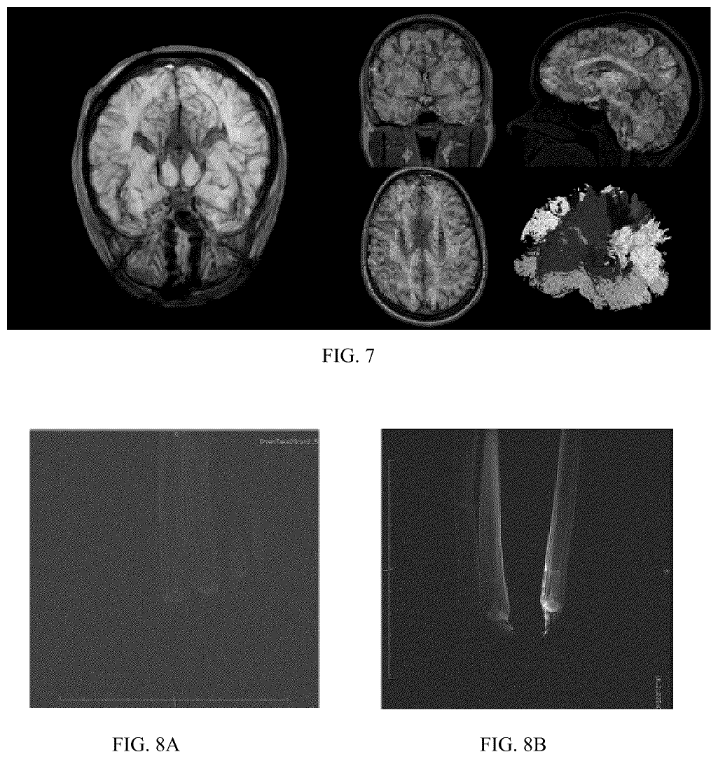

Referring to FIG. 7, this display screenshot illustrates a plurality of scans comprising a 3D/SWI scan and a tractography scan, in accordance with an embodiment of the present disclosure. The 3D/SWI scan is shown on the left side of the image. The tractography scan is shown on the right-side image of image, wherein fiber tractography and white matter segmentation of a human subject are shown, wherein a coronal overlay 71, a sagittal overlay 72, an axial overlay 73, and a fiber tract overlay 74 on a T1 weighted and white matter segmentation are shown.

### Phase 3: Intelligent DBS Coil

Referring to FIGS. 8A and 8B, together, these display screenshots illustrate a resolution, contrast, and definition comparison between an image acquired by using a\a head-coil and an image acquired by using a local, 2-channel, coil, in accordance with an embodiments of the present disclosure. FIG. 8A shows an MRI image of surgical instrument acquired by using only a head-coil and a field strength of 1.5 T; and FIG. 8B shows an MRI image of surgical instrument acquired by using only a local, 2-channel, coil and a field strength of 1.5 T.

The integrated system S is configured to direct guidance, via the guidance module, of electrodes, e.g., of the probe 250, by using enhanced imaging with MRI-micro-coils 80 coupled with, such as embedded in, the medical instrument, such as an insertion device, e.g., a DBS probe. The MRI device 400, e.g., the MRI device, is configured to operate with the MRI-micro-coils 80. The MRI-micro-coils 80 comprise an enhanced signal-to-noise (SNR) in a range of approximately 5 cm to approximately 30 cm, whereby a speed of real-time imaging is decreased over related art techniques, and whereby imaging resolution of the targets is increased over the related art. At least one of the probe 250 and the MRI micro-coils 80 are at least one of disposable and universal for all medical procedures. At least one of the probe 250 and the MRI micro-coils 80 is configured for use in at least one of the following medical procedures: biopsy, shunt, port, and other neurological procedures.

Referring to FIG. 9, this schematic diagram illustrates an integrated system S for supporting minimally invasive surgery, in accordance with an embodiment of the present disclosure. The system S comprises a control module 310 (FIG. 3), configurable by a set of executable instructions storable in relation to a non-transient memory device (not shown), to: receive at least one initial MRI image of anatomy from an MRI imaging device, e.g., the MRI device 400, as indicated by block 901; transmit the at least one initial MRI image to a planning module 301 configured to determine a surgical plan by using a tractography algorithm, as indicated by block 902; receive the surgical plan from the planning module 301, as indicated by block 903; transmit the surgical plan to a guidance module 303 configured to operate with a medical instrument, e.g., a DBS probe 250, and the MRI imaging device, the medical instrument configured to couple with at least one MRI micro-coil, e.g., at least one MRI-micro-coil 80, and the MRI imaging device configured to operate with the at least one MRI micro-coil, as indicated by block 904; and receive, in real-time, at least one subsequent MRI image of the medical instrument in relation to the anatomy from the MRI imaging device operating with the at least one MRI micro-coil during guidance of the medical instrument, as indicated by block 905.

Still referring to FIG. 9, in the system S, the control module 310 is further configurable, by the set of instructions, to: transmit, in real-time, the at least one subsequent MRI image of the medical instrument in relation to the anatomy to the planning module 301 further configured to update the surgical plan by using the tractography algorithm, whereby an updated surgical plan is providable, as indicated by block 906; receive, in real-time, the updated surgical plan from the planning module 301, as indicated by block 907; transmit, in real-time, the updated surgical plan to the guidance module 303, as indicated by block 908; receive, in real-time, at least one other subsequent MRI image of the medical instrument in relation to the anatomy from the MRI imaging device operating with the at least one MRI micro-coil during guidance of the medical instrument, as indicated by block 909; transmit the at least one subsequent MRI image to an external device for review, as indicated by block 910; and transmit the at least one other subsequent MRI image to an external device (not shown) for review, as indicated by block 911.

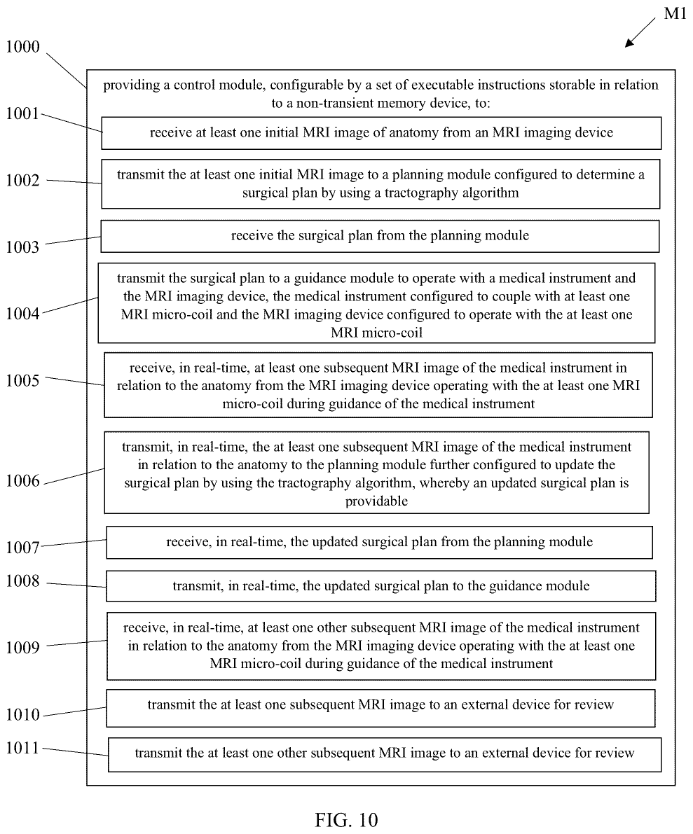

Referring to FIG. 10, this flow diagram illustrates a method Ml of providing an integrated system S for supporting minimally invasive surgery, in accordance with an embodiment of the present disclosure. The method M1 comprises providing a control module 310, configurable by a set of executable instructions storable in relation to a non-transient memory device (not shown), as indicated by block 1000, to: receive at least one initial MRI image of anatomy from an MRI imaging device, e.g., the MRI device 400, as indicated by block 1001; transmit the at least one initial MRI image to a planning module 301 configured to determine a surgical plan by using a tractography algorithm, as indicated by block 1002; receive the surgical plan from the planning module 301, as indicated by block 1003; transmit the surgical plan to a guidance module 303 configured to operate with a medical instrument, e.g., a DBS probe 250, and the MRI imaging device, the medical instrument configured to couple with at least one MRI micro-coil, e.g., at least one MRI-micro-coil 80, and the MRI imaging device configured to operate with the at least one MRI micro-coil, as indicated by block 1004; and receive, in real-time, at least one subsequent MRI image of the medical instrument in relation to the anatomy from the MRI imaging device operating with the at least one MRI micro-coil during guidance of the medical instrument, as indicated by block 1005.

Still referring to FIG. 10, in the method M1, providing the control module 310, as indicated by block 1000, comprises providing the control module 310 as further configurable, by the set of executable instructions, to at least one of: transmit, in real-time, the at least one subsequent MRI image of the medical instrument in relation to the anatomy to the planning module 301 further configured to update the surgical plan by using the tractography algorithm, whereby an updated surgical plan is providable, as indicated by block 1006; receive, in real-time, the updated surgical plan from the planning module 310, as indicated by block 1007; transmit, in real-time, the updated surgical plan to the guidance module 303, as indicated by block 1008; receive, in real-time, at least one other subsequent MRI image of the medical instrument in relation to the anatomy from the MRI imaging device operating with the at least one MRI micro-coil during guidance of the medical instrument, as indicated by block 1009; transmit the at least one subsequent MRI image to an external device (not shown) for review, as indicated by block 1010; and transmit the at least one other subsequent MRI image to an external device for review, as indicated by block 1011.

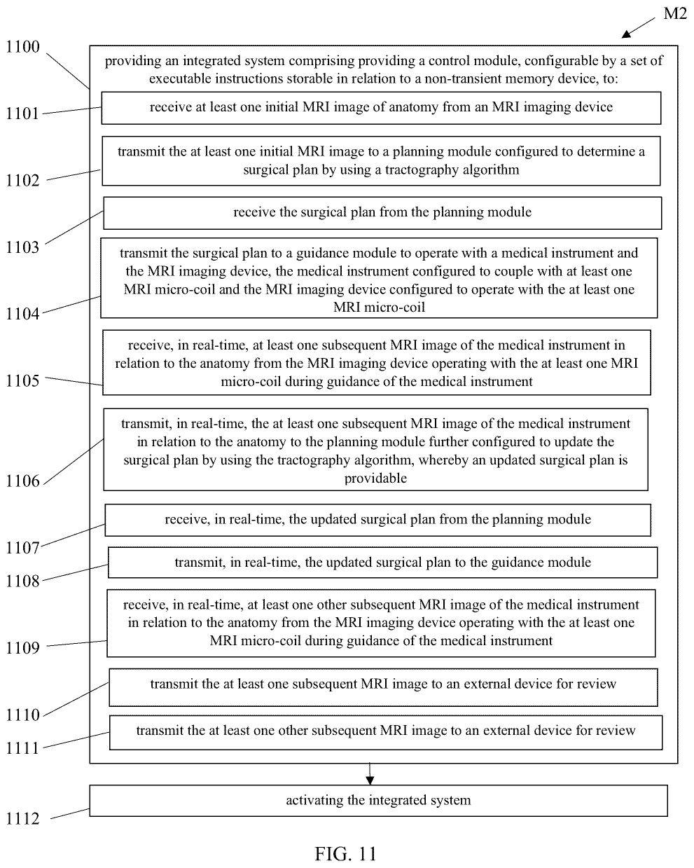

Referring to FIG. 11, this flow diagram illustrates a method M2 of minimizing surgical invasiveness, by way of a system S, in accordance with an embodiment of the present disclosure. The method M2 comprises: providing a control module 310, configurable by a set of executable instructions storable in relation to a non-transient memory device (not shown), as indicated by block 1100, to: receive at least one initial MRI image of anatomy from an MRI imaging device, e.g., the MRI device 400, as indicated by block 1101; transmit the at least one initial MRI image to a planning module 301 configured to determine a surgical plan by using a tractography algorithm, as indicated by block 1102; receive the surgical plan from the planning module 301, as indicated by block 1103; transmit the surgical plan to a guidance module 303 configured to operate with a medical instrument, e.g., a DBS probe 250, and the MRI imaging device, the medical instrument configured to couple with at least one MRI micro-coil, e.g., at least one MRI-micro-coil 80, and the MRI imaging device configured to operate with the at least one MRI micro-coil, as indicated by block 1104; and receive, in real-time, at least one subsequent MRI image of the medical instrument in relation to the anatomy from the MRI imaging device operating with the at least one MRI micro-coil during guidance of the medical instrument, as indicated by block 1105; and activating the integrated system, as indicated by block 1112.

Still referring to FIG. 11, in the method M2, providing the control module 310, as indicated by block 1100, comprises providing the control module 310 as further configurable, by the set of executable instructions, to at least one of: transmit, in real-time, the at least one subsequent MRI image of the medical instrument in relation to the anatomy to the planning module 301 further configured to update the surgical plan by using the tractography algorithm, whereby an updated surgical plan is providable, as indicated by block 1106; receive, in real-time, the updated surgical plan from the planning module 310, as indicated by block 1107; transmit, in real-time, the updated surgical plan to the guidance module 303, as indicated by block 1108; receive, in real-time, at least one other subsequent MRI image of the medical instrument in relation to the anatomy from the MRI imaging device operating with the at least one MRI micro-coil during guidance of the medical instrument, as indicated by block 1109; transmit the at least one subsequent MRI image to an external device (not shown) for review, as indicated by block 1110; and transmit the at least one other subsequent MRI image to an external device for review, as indicated by block 1111.

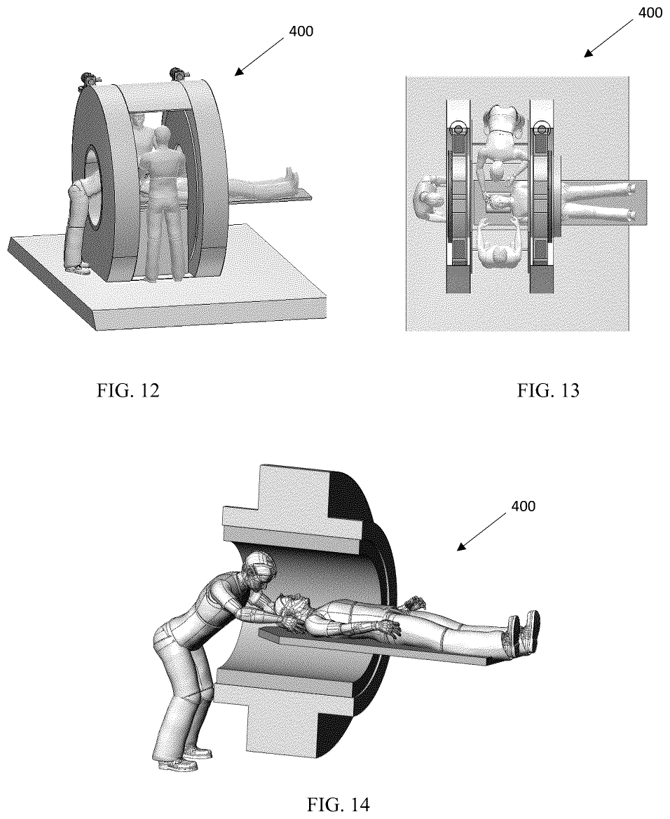

Referring to FIG. 12, this diagram illustrates, in a perspective view, an MRI device 400 operable with an integrated system S, in accordance with an embodiment of the present disclosure.

Referring to FIG. 13, this diagram illustrates, in a top view, an MRI device 400 operable with an integrated system, in accordance with an embodiment of the present disclosure.

Referring to FIG. 14, this diagram illustrates, in a cutaway perspective view, an MRI device 400 operable with an integrated system. in accordance with an embodiment of the present disclosure.

Referring back to FIGS. 1-14, benefits of the present disclosure embodiments over the related art include, but are not limited to, an integration of preoperative navigation strategies, intraoperative imaging strategies, intraoperative guidance strategies, and high-definition imaging strategies for better minimizing surgical invasiveness, especially for intracranial procedures, and providing integrated surgical strategies for a larger population of patients in need.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become obvious to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

What is claimed:

1. An integrated system for supporting minimally invasive surgery, the system comprising a control module, configurable by a set of executable instructions storable in relation to a non-transient memory device, to:
receive at least one initial MRI image of anatomy from an MRI imaging device;
transmit the at least one initial MRI image to a planning module configured to determine a surgical plan by using a tractography algorithm;
receive the surgical plan from the planning module;
transmit the surgical plan to a guidance module configured to operate with a medical instrument and the MRI imaging device, the medical instrument configured to couple with at least one MRI micro-coil, and the MRI imaging device configured to operate with the at least one MRI micro-coil; and
receive, in real-time, at least one subsequent MRI image of the medical instrument in relation to the anatomy from the MRI imaging device operating with the at least one MRI micro-coil during guidance of the medical instrument.

2. The system of claim 1, wherein the control module is further configurable, by the set of instructions, to transmit, in real-time, the at least one subsequent MRI image of the medical instrument in relation to the anatomy to the planning module further configured to update the surgical plan by using the tractography algorithm, whereby an updated surgical plan is providable.

3. The system of claim 2, wherein the control module is further configurable, by the set of instructions, to receive, in real-time, the updated surgical plan from the planning module.

4. The system of claim 3, wherein the control module is further configurable, by the set of instructions, to transmit, in real-time, the updated surgical plan to the guidance module.

5. The system of claim 4, wherein the control module is further configurable, by the set of instructions, to receive, in real-time, at least one other subsequent MRI image of the medical instrument in relation to the anatomy from the MRI imaging device operating with the at least one MRI micro-coil during guidance of the medical instrument.

6. The system of claim 1, wherein the control module is further configurable, by the set of instructions, to transmit the at least one subsequent MRI image to an external device for review.

7. The system of claim 5, wherein the control module is further configurable, by the set of instructions, to transmit the at least one other subsequent MRI image to an external device for review.

8. A method of providing an integrated system for supporting minimally invasive surgery, the method comprising providing a control module, configurable by a set of executable instructions storable in relation to a non-transient memory device, to:
receive at least one initial MRI image of anatomy from an MRI imaging device;
transmit the at least one initial MRI image to a planning module configured to determine a surgical plan by using a tractography algorithm;
receive the surgical plan from the planning module;
transmit the surgical plan to a guidance module configured to operate with a medical instrument and the MRI imaging device, the medical instrument configured to couple with at least one MRI micro-coil, and the MRI imaging device configured to operate with the at least one MRI micro-coil; and
receive, in real-time, at least one subsequent MRI image of the medical instrument in relation to the anatomy from the MRI imaging device operating with the at least one MRI micro-coil during guidance of the medical instrument.

9. The method of claim 8, wherein providing the control module comprises providing the control module as further configurable, by the set of executable instructions, to transmit, in real-time, the at least one subsequent MRI image of the medical instrument in relation to the anatomy to the planning module further configured to update the surgical plan by using the tractography algorithm, whereby an updated surgical plan is providable.

10. The method of claim 9, wherein providing the control module comprises providing the control module as further configurable, by the set of executable instructions, to receive, in real-time, the updated surgical plan from the planning module.

11. The method of claim 10, wherein providing the control module comprises providing the control module as further configurable, by the set of executable instructions, to transmit, in real-time, the updated surgical plan to the guidance module.

12. The method of claim 11, wherein providing the control module comprises providing the control module as further configurable, by the set of executable instructions, to receive, in real-time, at least one other subsequent MRI image of the medical instrument in relation to the anatomy from the MRI imaging device operating with the at least one MRI micro-coil during guidance of the medical instrument.

13. The method of claim 8, wherein providing the control module comprises providing the control module as further configurable, by the set of executable instructions, to transmit the at least one subsequent MRI image to an external device for review.

12. method of claim 12, wherein providing the control module comprises providing the control module as further configurable, by the set of executable instructions, to transmit a tie at least one other subsequent MRI image to an external device for review.

15. A method of supporting minimally invasive surgery by way of an integrated system, the method comprising:
providing a control module, configurable by a set of executable instructions storable in relation to a non-transient memory device, to:
receive at least one initial MRI image of anatomy from an MRI imaging device;
transmit the at least one initial MRI image to a planning module configured to determine a surgical plan by using a tractography algorithm;
receive the surgical plan from the planning module;
transmit the surgical plan to a guidance module configured to operate with a medical instrument and the MRI imaging device, the medical instrument configured to couple with at least one MRI micro-coil, and the MRI imaging device configured to operate with the at least one MRI micro-coil; and
receive, in real-time, at least one subsequent MRI image of the medical instrument in relation to the anatomy from the MRI imaging device operating with the at least one MRI micro-coil during guidance of the medical instrument; and
activating the integrated system.

16. The method of claim 15, wherein providing the control module comprises providing the control module as further configurable, by the set of executable instructions, to transmit, in real-time, the at least one subsequent MRI image of the medical instrument in relation to the anatomy to the planning module further configured to update the surgical plan by using the tractography algorithm, whereby an updated surgical plan is providable.

17. The method of claim 16, wherein providing the control module comprises providing the control module as further configurable, by the set of executable instructions, to receive, in real-time, the updated surgical plan from the planning module.

18. The method of claim 17,
wherein providing the control module comprises providing the control module as further configurable, by the set of executable instructions, to transmit, in real-time, the updated surgical plan to the guidance module, and
wherein providing the control module comprises providing the control module as further configurable, by the set of executable instructions, to receive, in real-time, at least one other subsequent MRI image of the medical instrument in relation to the anatomy from the MRI imaging device operating with the at least one MRI micro-coil during guidance of the medical instrument.

19. The method of claim 15, wherein providing the control module comprises providing the control module as further configurable, by the set of executable instructions, to transmit the at least one subsequent MRI image to an external device for review.

20. The method of claim 18, wherein providing the control module comprises providing the control module as further configurable, by the set of executable instructions, to transmit the at least one other subsequent MRI image to an external device for review.
